(19) United States
(12) Patent Application Publication
(10) Pub. No.: US 2013/0121931 A1
(43) Pub. Date: May 16, 2013

(54) METHOD FOR CONTROLLING CONTINUOUSNESS OF DEODORANT EFFECT AFTER CONSUMPTION OF CHEWING GUM COMPOSITION AND A CHEWING GUM COMPOSITION

(71) Applicants: Masafumi YAMAI, Tokyo (JP); Kenichiro YANAGI, Tokyo (JP); Yoshiaki ADACHI, Tokyo (JP); Samantha HOLME, Whippany, NJ (US); Nobuhiro HANADA, Kanagawa (JP); Susumu IMAI, Kanagawa (JP)

(72) Inventors: Masafumi YAMAI, Tokyo (JP); Kenichiro YANAGI, Tokyo (JP); Yoshiaki ADACHI, Tokyo (JP); Samantha HOLME, Whippany, NJ (US); Nobuhiro HANADA, Kanagawa (JP); Susumu IMAI, Kanagawa (JP)

(21) Appl. No.: 13/312,137

(22) Filed: Dec. 6, 2011

Related U.S. Application Data
(60) Provisional application No. 61/559,219, filed on Nov. 14, 2011.

(51) Int. Cl. A61K 9/68; A61Q 11/00

(52) U.S. Cl. 424/48

(57) ABSTRACT

An object of the present invention is to provide a method for controlling the continuousness of deodorant effect after consumption of a chewing gum composition, and a chewing gum composition having excellent continuousness of the deodorant effect after consumption of the chewing gum composition. The method for controlling continuousness of the deodorant effect after consumption of the chewing gum composition, in which the chewing gum composition contains a biphenyl compounds-containing extract and a tannins-containing extract, includes the step of controlling the ratio of a blending amount of the tannins-containing extract to a blending amount of biphenyl compounds-containing extract. The chewing gum composition contains a biphenyl compounds-containing extract and a tannins-containing extract, in which the ratio by mass of the blending amount of the tannins-containing extract to the blending amount of the biphenyl compounds-containing extract is greater than 5.0.

### BACKGROUND OF THE INVENTION

1. Field of the Invention

The present invention relates to a technology for controlling the continuousness of deodorant effect after consumption of a chewing gum composition.

2. Related Art

Bad breath is mainly caused by volatile sulfur-containing compounds including methyl mercaptan, and gives displeasure to many people. To date, various deodorants or deodorizing substances have been developed and proposed for deodorizing the bad breath. Among these, a number of deodorants or deodorizing substances utilizing plant extract(s) have been proposed from the perspective of safety towards the human body (for example, see Japanese Unexamined Patent Application, Publication No. S57-204278).

Besides, a deodorant containing a plant extract as a constituent effective for deodorizing bad breath in combination with an oxidoreductase (see Japanese Unexamined Patent Application, Publication No. S63-309269), and a deodorant containing a specific plant extract in combination with an enzyme that oxidizes phenolic compounds (see Japanese Unexamined Patent Applications, Publication No. H10-212221 and 2010-158375) have been proposed as a deodorant having sufficient deodorizing power and high safety from a use aspect.

### SUMMARY OF THE INVENTION

However, it is merely the degree of the deodorizing power that has been pursued to date. For example, in the technical field of chewing gums, consumption of a chewing gum containing deodorizing substances can afford the deodorant effect during consumption of the chewing gum; however, it has not been extensively investigated how long the deodorant effect persists after consumption of the chewing gum. Thus, sufficient deodorant effect may not be achieved without continued consumption of the chewing gum.

The present invention has been made in view of such a problem and has an object of providing a method for controlling the continuousness of the deodorant effect after consumption of a chewing gum composition, and a chewing gum composition having excellent continuousness of the deodorant effect after consumption.

The present inventors have found that, in chewing gums containing a biphenyl compounds-containing extract and a tannins-containing extract, the ratio of the blending amount of the tannins-containing extract to the blending amount of biphenyl compounds-containing extract correlates with the continuousness of the deodorant effect after consumption of the chewing gum composition, and thus, based on this fact, achieved the present invention. More specifically, the present invention provides the following.

A first aspect of the present invention provides a method for controlling the continuousness of deodorant effect after consumption of a chewing gum composition, the chewing gum composition containing a biphenyl compounds-containing extract and a tannins-containing extract, the method including the step of controlling a ratio of a blending amount of the tannins-containing extract to a blending amount of the biphenyl compounds-containing extract.

In a second aspect of the method according to the first aspect of the present invention, the deodorant effect is at a level above an arithmetic average of a deodorant effect of each extract.

In a third aspect of the method according to either the first or second aspect of the present invention, the ratio by mass of the blending amount of the tannins-containing extract to the blending amount of the biphenyl compounds-containing extract is greater than 5.0.

A fourth aspect of the method according to any one of the first to third aspects of the present invention, further includes blending a peroxidase into the chewing gum composition.

In a fifth aspect of the method according to any one of the first to fourth aspects, the biphenyl compounds-containing extract includes a thyme extract, and the tannins-containing extract includes a Quercus salicina Blume extract.

A sixth aspect of the present invention provided is a chewing gum composition containing a biphenyl compounds-containing extract and a tannins-containing extract,

in which a ratio by mass of a blending amount of the tannins-containing extract to a blending amount of the biphenyl compounds-containing extract is greater than 1.0.

A seventh aspect of the chewing gum composition according to the sixth aspect of the present invention, further includes a peroxidase.

In an eighth aspect of the chewing gum composition according to either the sixth or seventh aspect of the present invention, the biphenyl compounds-containing extract includes a thyme extract, and the tannins-containing extract includes a Quercus salicina Blume extract.

According to the present invention, controlling the continuousness of the deodorant effect after consumption of the chewing gum composition can be achieved by controlling the ratio of the blending amount of the tannins-containing extract to the blending amount of biphenyl compounds-containing extract.

### BRIEF DESCRIPTION OF THE DRAWINGS

FIG. 1 is a graph showing the continuousness of the deodorant effect after consumption of the chewing gum composition according to an example of the present invention; and

FIG. 2 is a graph showing expected continuousness of the deodorant effect after consumption of the chewing gum composition.

### DETAILED DESCRIPTION OF THE INVENTION

Hereinbelow, an embodiment according to the present invention will be described, but the present invention should not be construed to be limited thereto.

In the present invention, a method for controlling the continuousness of deodorant effect after consumption of a chewing gum composition, in which the chewing gum composition contains a biphenyl compounds-containing extract and a tannins-containing extract, includes the step of controlling the ratio of a blending amount of the tannins-containing extract to a blending amount of the biphenyl compounds-containing extract. Since the deodorant effect during consumption of a chewing gum depends on the amount of the deodorizing substances leaching from the chewing gum to saliva, the effect can be controlled by the leachability of the deodorizing substances into saliva or the blending amount of the deodorizing substances in the chewing gum composition. After consumption of the chewing gum, however, the chewing gum, which was a source of deodorizing substances, has been removed from the oral cavity, and the deodorizing substances thus leached has been swallowed along with the saliva from the oral cavity to the stomach. Consequently, the continuousness of the deodorant effect after consumption is not expected to correlate well to the leachability of the deodorizing substances into saliva or the blending amount of the deodorizing substances in the chewing gum composition, and the control thereof is difficult.

The present inventors have found that the ratio of the blending amount of the tannins-containing extract to the blending amount of biphenyl compounds-containing extract correlates to the continuousness of the deodorant effect after consumption of the chewing gum composition containing the same. The above finding is surprising, since it is unclear in current state of the art whether or not there is a significant difference between the biphenyl compounds-containing extract and the tannins-containing extract in the continuousness of the deodorant effect after consumption. According to the present invention, controlling the continuousness of the deodorant effect after consumption of the chewing gum composition can be achieved by controlling the ratio of the blending amount of the tannins-containing extract to the blending amount of the biphenyl compounds-containing extract. Here, it should be noted that the continuousness of the deodorant effect after consumption can be controlled by the ratio as described above, i.e., a parameter other than the leachability of the deodorizing substances into saliva or the blending amount, which fact means that the continuousness of the deodorant effect can be optimized independently of the deodorant effect during consumption of the chewing gum composition.

The biphenyl compounds-containing extract includes, but is not limited to, a thyme extract, and one or more biphenyl compounds-containing extracts can be employed. The extracts of this kind contain deodorizing ingredients having a common basic skeleton, and hence have common retentivity in the oral cavity and common mechanism for inducing the deodorant effect, and cooperate with the tannins-containing extract to induce similar continuousness of the deodorant effect after consumption. The biphenyl compounds-containing extract preferably, without specific limitation, includes a thyme extract. The blending amount of the biphenyl compounds-containing extract is not specifically limited, but is generally in the range of 0.0001 to 10% by weight, preferably 0.005 to 1% by weight of the chewing gum composition.

The tannins-containing extract includes, but is not limited to, a Quercus salicina Blume extract, a Rubus suavissimus S. Lee extract, and a persimmon extract, and one or more tannins-containing extracts can be employed. The extracts of this kind contain deodorizing ingredients having a common basic skeleton, and hence have common retentivity in the oral cavity and common mechanism for inducing the deodorant effect, and cooperate with the biphenyl compounds-containing extract to induce similar continuousness of the deodorant effect after consumption. The tannins-containing extract preferably, without specific limitation, includes a Quercus salicina Blume extract. The blending amount of the tannins-containing extract is not specifically limited, but is generally in the range of 0.0001 to 10% by weight, preferably 0.005 to 1% by weight of the chewing gum composition.

It is noted that in the present invention, the phrase “controlling the continuousness of the deodorant effect after consumption of the chewing gum composition” encompasses, but is not limited to, an aspect in which a given chewing gum composition differs from conventional chewing gum compositions in the ratio of the blending amount of the tannins-containing extract to the blending amount of the biphenyl compounds-containing extract, and a sign, an advertisement or the like regarding the continuousness of the deodorant effect after consumption is associated with the chewing gum composition.

The deodorant effect after consumption provided by a combination of the biphenyl compounds-containing extract and the tannins-containing extract preferably persists at a level above the arithmetic average of the deodorant effect of each extract. A period of time in which the deodorant effect is at a level above the arithmetic average is not necessarily the entire period of time after consumption, but may be merely a limited period of time (for example, 30, 60, 90, or 120 minutes after the end of the consumption). It should be noted that the deodorant effect is determined by the total concentration of volatile sulfur-containing compounds (VSC), i.e., hydrogen sulfide, methyl mercaptan, and dimethyl sulfide, in the expired breath. More specifically, greater deodorant effect can be identified by lower concentration of the VSC in the expired breath.

The ratio by mass of the blending amount of the tannins-containing extract to the blending amount of the biphenyl compounds-containing extract is not specifically limited, but preferably is greater than 5.0. This facilitates the achievement of excellent continuousness of the deodorant effect after consumption. More preferably, the ratio described above is no less than 5.5, and still more preferably, no less than 6.0, no less than 6.5, no less than 7.0, or no less than 7.5. Furthermore, the ratio is preferably, but not limited to, no greater than 50, more preferably no greater than 40, or no greater than 25. Nonetheless, the ratio may be no greater than 5.0.

Preferably, the method according to the present invention further includes the step of blending a peroxidase into the chewing gum composition. This facilitates the achievement of excellent continuousness of the deodorant effect after consumption. However, the benefit of the present invention afforded by changing the ratio of the tannins-containing extract to the biphenyl compounds-containing extract can be obtained without blending the peroxidase.

The present invention further provides a chewing gum composition containing a biphenyl compounds-containing extract and a tannins-containing extract, in which the ratio by mass of the blending amount of the tannins-containing extract to the blending amount of the biphenyl compounds-containing extract is greater than 5.0. Such a chewing gum composition has excellent continuousness of the deodorant effect after consumption, as described above.

The blending amount of each extract is not specifically limited, but is generally 0.0001 to 10% by weight, preferably 0.005 to 1% by weight of the chewing gum composition.

Each extract is prepared by treating suitable raw plant materials with an extraction solvent. There is no particular limitation to the extraction solvent, but polar solvents are generally preferred, and may include, for example, water, lower aliphatic alcohols, or mixtures thereof. The lower aliphatic alcohols includes, for example, ethanol, propanol, isopropanol, butanol, 1,4-butylene glycol, ethylene glycol, propylene glycol, isopropylene glycol or the like. Among these, water is preferred. When aqueous lower aliphatic alcohols are used as the extraction solvent, the amount of the alcohol contained therein may be 10 to 90% by mass.

The water as the extraction solvent includes, but is not limited to, for example, pure water, tap water, and well water, or those obtained by applying to the water of the type described above various treatments such as ion exchange, membrane treatment, filtration, heat sterilization, distillation, pH adjustment or the like.

Extraction conditions can be determined as appropriate, depending on the nature of the raw plant materials. An example of the extraction conditions include adding 3 to 20-fold amount by weight of a polar solvent to dried leaves or crushed sticks of the raw plant materials or the like, impregnating or stirring the dried leaves or crushed sticks of the raw plant materials or the like with the polar solvent, and thereby performing the extraction. The extraction time is preferably in the range of 30 minutes to one day, and the extraction temperature is preferably in the range of room temperature to 100° C.

The extraction operation may be performed only once, and the extraction operation may be repeated by adding a second portion of the polar solvent to the residue of the leaves or crushed sticks of the raw plant materials or the like recovered after the first extraction operation. The extraction liquids obtained in the respective extraction operations may be combined together into one entire extraction liquid, or may be used separately.

There is no particular limitation to the form of the plant extract obtained from the raw plant materials, and for example, the extraction liquid per se, or diluted solutions, concentrated solutions or lyophilizates thereof or substances obtained by purifying the extraction liquid through a fractionation or purification process, or the like can be employed.

When the raw plant materials are extracted with the polar solvent, a nonpolar solvent (for example, hexanes, benzene, toluene or the like) may be preliminarily added to the raw plant materials to promote the dissolution and elimination of fat-soluble components in the raw plant materials.

In order to enhance the functionalities of the extract thus obtained, the extract may be subjected to purification treatments such as activated carbon treatment, adsorption resin treatment, ion exchange resin treatment or the like alone or in combination with each other, as appropriate. Furthermore, for easy handling of the extract, the extract may be further processed, e.g., formulated.

Furthermore, the chewing gum composition according to the present invention preferably, without specific limitation, contains a peroxidase. The peroxidase may be obtained from a squeeze of horseradish (Armoracia rusticana), radish (Rahpauns acanthiformis) or cucumber (Cucumis sativus), or obtained by extracting a culture fluid of filamentous fungi (Alternaria, Aspergillus oryzae, Coprinus cinereus, or Oidiodendron) or bacteria (Bacillus) with water at low to room temperature, or by concentrating and purifying the culture fluid at low to room temperature. Peroxidases are readily available, and commercially available peroxidases may be employed.

In the chewing gum composition according to the present invention, it is preferable, without limitation, that the biphenyl compounds-containing extract includes a thyme extract and the tannins-containing extract includes a Quercus salicina Blume extract.

The chewing gum composition according to the present invention may contain, in addition to the deodorizing substances described above, optional components including any gum base, sweeteners, flavors, food colors, functional ingredients (for example, refreshing agents, throat-soothing agents, spices, warming agents, tooth whitening agents, vitamins, minerals, caffeine, pharmacological agents) and mixtures thereof.

The chewing gum composition according to the present invention can be formed alone into chewing gum products. Alternatively, chewing gum products in which a center-filling composition is surrounded by the chewing gum composition according to the present invention, or in which the chewing gum composition according to the present invention is, for example, coated with an outer layer of sugarcoating or the like can be produced. The center-filling composition and the sugarcoating or the like can be of any composition.

### EXAMPLES

(Preparation of Quercus Salicina Blume Extract)

Three hundred grams of distilled water was added to 50 g of dried and crushed leaves (including sticks) of Quercus salicina Blume, and the mixture was heated to reflux for 2 h. The resultant extract mixture was allowed to cool at room temperature, and filtered to separate the extraction liquid from the residue. Again, 300 g of distilled water was added to the residue, and then the mixture was heated to reflux, allowed to cool and filtered in the same manner as described above. The extraction operation was performed three times, and all of the extraction liquids obtained in the respective operations were combined, and the combined liquid was concentrated to dryness under reduced pressure to yield a Quercus salicina Blume extract.

(Preparation of Thyme Extract)

Three hundred grams of 50% (v/v) aqueous alcohol was added to 30 g of dried leaves of thyme, and the mixture was heated to reflux at 80° C. for 3 h. The resultant extract mixture was allowed to cool at room temperature, and filtered to separate the extraction liquid from the residue. Again, 300 g of 50% (v/v) aqueous alcohol was added to the residue, and then the mixture was heated to reflux, allowed to cool and filtered in the same manner as described above. The extraction operation was performed three times, and all of the extraction liquids obtained in the respective operations were combined, and the combined liquid was concentrated to dryness under reduced pressure to yield a thyme extract.

(Preparation of Chewing Gum Composition)

A chewing gum composition was prepared in accordance with the following formulation:

### Comparative Example

### Placebo Gum

| | |
|---|---|
| gum base | 35 g |
| maltitol | 55 g |
| xylitol | 5 g |
| gum flavor | 1 g |

### Reference Example

### Quercus Salicina Blume Extract/Thyme Extract=5.0)

| | |
|---|---|
| Quercus salicina Blume extract | 0.035 g |
| thyme extract | 0.007 g |
| peroxidase | 0.0001 g |
| gum base | 35 g |
| maltitol | 55 g |
| xylitol | 5 g |
| gum flavor | 1 g |

### Example

### Quercus Salicina Blume Extract/Thyme Extract=12.5)

| | |
|---|---|
| Quercus salicina Blume extract | 0.091 g |
| thyme extract | 0.0073 g |
| peroxidase | 0.0001 g |
| gum base | 35 g |
| maltitol | 55 g |
| xylitol | 5 g |
| gum flavor | 1 g |

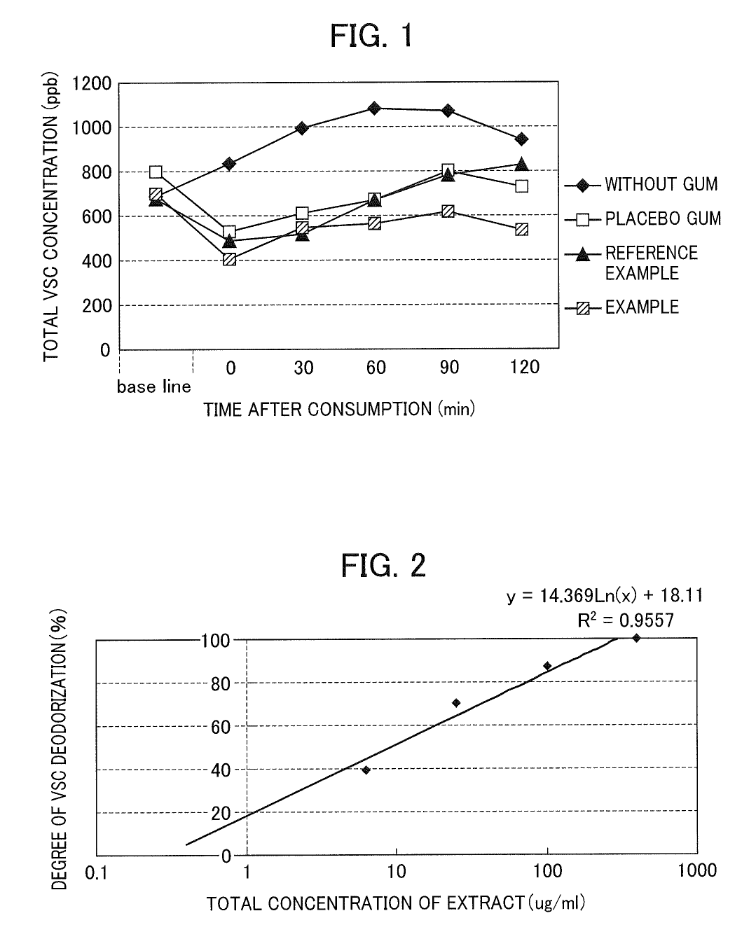

Each chewing gum composition was consumed by 37 healthy subjects for 20 minutes. Control group was kept waiting for 20 minutes, without consuming any chewing gum. A breath sample was collected from the subjects before consumption of the chewing gum composition (designated as “base line” in FIG. 1) and 0, 30, 60, 90, 120 minutes after removal of the chewing gum composition from the oral cavity, and the concentration of hydrogen sulfide, methyl mercaptan, and dimethyl sulfide in the breath sample was measured by gas chromatography through direct injection of the breath sample. Degree of VSC deodorization in the breath sample was calculated according to the following equation. Total concentration of volatile sulfur-containing compounds (VSC) at each time point is shown in FIG. 1.

Degree of VSC deodorization (%)=(B−T)/B×100

wherein:

B: Total concentration (ppb) of VSC in the breath sample before consumption of the chewing gum composition;

T: Total concentration (ppb) of VSC in the breath sample after consumption of the chewing gum composition.

As shown in FIG. 1, each of the chewing gum examined gave significant deodorant effect immediately after consumption of the chewing gum, as compared to the case without any chewing gum, and the degree of the deodorant effect for each chewing gum was comparable. However, the deodorant effect of the chewing gums of Comparative Example and Reference Example gradually disappeared with the time elapsed after consumption, and VSC values returned to the same level as prior to the consumption. In contrast, the deodorant effect after consumption of the chewing gum of Example persisted over time; this chewing gum solely gave significant deodorant effect even at 120 minutes after consumption of the chewing gum, compared to the control group (i.e., without any chewing gum).

This result indicates that the continuousness of the deodorant effect after consumption can be controlled by the ratio of the blending amount of the Quercus salicina Blume extract to the blending amount of the thyme extract. The chewing gum of Example is somewhat higher in total blending amount of the thyme extract and the Quercus salicina Blume extract than in Reference Example. However, it is not presumable that the difference in the continuousness of the deodorant effect after consumption is attributed to the difference in the total blending amount of the extracts, since after consumption of the chewing gum, which was a source of the deodorizing substances, the chewing gum has been removed from the oral cavity, and the deodorizing substances thus leached has been swallowed along with the saliva from the oral cavity to the stomach.

### Testing Example 1

### Effect of the Amount of the Extracts

As described above, it is not presumable that the difference in the continuousness of the deodorant effect after consumption is attributed to the difference in the total blending amount of the extracts, and the following Testing Example was carried out to verify the presumption. The condition of this Testing Example simulates the condition within the oral cavity during consumption of the chewing gum.

To a sealable test tube of 10 ml volume was added 1.9 ml of a 0.1 M phosphate buffer, and a mixed extract of the Quercus salicina Blume extract and the thyme extract in the ratio of 5:1 was dissolved therein at the final concentration of 0.000094% to 0.006% (w/v), and then a peroxidase (specific activity: 100 U/mg) was added at the final concentration of 0.00003% (w/v). Then, 0.1 ml of a 2 ppm methyl mercaptan solution was added, and immediately thereafter the test tube was sealed. Each test sample was kept at 37° C. for 30 minutes, 2 ml of the gas in the headspace of the test tube was taken, and the concentration (ppb) of methyl mercaptan in the gas was measured by gas chromatography. The results are shown in FIG. 2.

Degree of deodorization (%)=(C−S)/C×100

wherein:

C: Concentration (ppb) of methyl mercaptan in the headspace for control group;

S: Concentration (ppb) of methyl mercaptan in the headspace for deodorizing substances addition test group.

As shown in FIG. 2, a certain correlation equation was found to be satisfied between the concentration of the extract and the degree of deodorization. The amount of the extracts in Example is 2.34-fold (0.0983/0.042) relative to that in Reference Example, and, based on this correlation equation, the degree of deodorization for Example (30.3%) was expected to be 12.22% higher than the degree of deodorization for Reference Example (18.1%) (Table 1).

### [Table 1]

However, as shown in FIG. 2, the difference in the degree of deodorization between Example and Reference Example at respective time points after consumption was greater than the expected value of 12.22%. This shows that the change of the deodorant effect after consumption of the chewing gum is independent of the ratio of the amount of the extracts, and is greater than expected on the basis of the deodorant effect during consumption the chewing gum.

### [Table 2]

### Testing Example 2

### Effect of the Peroxidase

In order to confirm that the difference in the deodorant effect after consumption caused by the control of the ratio of the extracts can be produced irrespective of the presence or absence of the peroxidase, the following Testing Example was carried out.

To a sealable vial of 50 ml volume was added 9.9 ml of a 0.1 M phosphate buffer, a mixed extract of the Quercus salicina Blume extract and the thyme extract in the ratio of 1:2 or 2:1 was dissolved therein at the final concentration of 0.000150 (w/v), and then a peroxidase (specific activity: 100 U/mg) was added at the final concentration of 0.000030 (designated as “peroxidase (+)”) or 0.00000% (designated as “peroxidase (−)”) (w/v). Then, 0.1 ml of a 10 ppm methyl mercaptan solution was added, and immediately thereafter the vial was sealed. Each test sample was kept at 37° C. for 30-120 minutes, 2 ml of the gas in the headspace of the vial was taken, and the concentration (ppb) of methyl mercaptan in the gas was measured by gas chromatography. The definition of the degree of deodorization is the same as in Testing Example 1. The results are summarized in Table 3.

### [Table 3]

As shown in Table 3, it is clear that by controlling the ratio of the amount of the extracts, an incremental increase in the degree of deodorization over a prolonged time resulted, and this effect can be produced irrespective of the presence or absence of the peroxidase. Still, the inclusion of the peroxidase is clearly preferred in terms of the large absolute value and large increment of the degree of deodorization.

1. A method for controlling continuousness of deodorant effect after consumption of a chewing gum composition, the chewing gum composition comprising a biphenyl compounds-containing extract and a tannins-containing extract, the method comprising the step of controlling a ratio by mass of a blending amount of the tannins-containing extract to a blending amount of biphenyl compounds-containing extract; wherein the ratio by mass of a blending amount of the tannins-containing extract to a blending amount of the biphenyl compounds-containing extract is greater than 5.0.

2. The method of claim 1, wherein the deodorant effect is at a level above an arithmetic average of a deodorant effect of each of the extract.

3-4. (canceled)

5. The method of claim 4, further comprising blending a peroxidase into the chewing gum composition.

6. The method of claim 3, further comprising blending a peroxidase into the chewing gum composition.

7. The method of claim 2, further comprising blending a peroxidase into the chewing gum composition.

8. The method of claim 1, further comprising blending a peroxidase into the chewing gum composition.

9. The method of claim 8, wherein the biphenyl compounds-containing extract comprises a thyme extract, and the tannins-containing extract comprises a Quercus salicina Blume extract.

10. The method of claim 7, wherein the biphenyl compounds-containing extract comprises a thyme extract, and the tannins-containing extract comprises a Quercus salicina Blume extract.

11. The method of claim 6, wherein the biphenyl compounds-containing extract comprises a thyme extract, and the tannins-containing extract comprises a Quercus salicina Blume extract.

12. The method of claim 5, wherein the biphenyl compounds-containing extract comprises a thyme extract, and the tannins-containing extract comprises a Quercus salicina Blume extract.

13. The method of claim 4, wherein the biphenyl compounds-containing extract comprises a thyme extract, and the tannins-containing extract comprises a Quercus salicina Blume extract.

14. The method of claim 3, wherein the biphenyl compounds-containing extract comprises a thyme extract, and the tannins-containing extract comprises a Quercus salicina Blume extract.

15. The method of claim 2, wherein the biphenyl compounds-containing extract comprises a thyme extract, and the tannins-containing extract comprises a Quercus salicina Blume extract.

16. The method of claim 1, wherein the biphenyl compounds-containing extract comprises a thyme extract, and the tannins-containing extract comprises a Quercus salicina Blume extract.

17. A chewing gum composition comprising a biphenyl compounds-containing extract and a tannins-containing extract, wherein a ratio by mass of a blending amount of the tannins-containing extract to a blending amount of the biphenyl compounds-containing extract is greater than 5.0.

18. The composition of claim 17, further comprising a peroxidase.

19. The composition of claim 18, wherein the biphenyl compounds-containing extract comprises a thyme extract, and the tannins-containing extract comprises a Quercus salicina Blume extract.

20. The composition of claim 17, wherein the biphenyl compounds-containing extract comprises a thyme extract, and the tannins-containing extract comprises a Quercus salicina Blume extract.
